(19) United States
(12) Patent Application Publication
(10) Pub. No.: US 2021/0094054 A1
(43) Pub. Date: Apr. 1, 2021

(54) Hypochlorous Acid And Complementary Substance Dispensing System

(71) Applicant: Mark D. Noar, Owings Mills, MD (US)

(72) Inventor: Mark D. Noar, Owings Mills, MD (US)

(21) Appl. No.: 17/029,111

(22) Filed: Sep. 23, 2020

Related U.S. Application Data
(60) Provisional application No. 62/907,864, filed on Sep. 30, 2019.

(51) Int. Cl. B05B 11/00; A01N 59/00; A61K 8/9794; A61K 8/27; A61Q 19/00; A61K 8/20; A61K 31/60; A61K 31/19; A61K 33/20

(52) U.S. Cl. CPC B05B 11/3081; A01N 59/00; A61K 8/9794; A61K 8/27; A61Q 19/00; B05B 11/3057; A61K 31/60; A61K 31/19; A61K 33/20; B05B 11/3045; A61K 8/20

(57) ABSTRACT

A hypochlorous acid dispensing system having a first cartridge or compartment containing pure hypochlorous acid. A second cartridge or compartment, separate from the first cartridge or compartment, contains a substance that can be mixed with or used with the pure hypochlorous acid. A dispensing structure is associated with each of the first and second cartridges or compartments and is constructed and arranged to cause dispensing of the pure hypochlorous acid and the substance separately or simultaneously. A kit including at least one of the first cartridges and at last one of the second cartridges is also provided.

### BACKGROUND

Hypochlorous acid (HOCl), also known as electrolyzed water, is a known organic disinfectant and is typically packaged for industrial use to disinfect surfaces in restrooms and hospitals. Hypochlorous acid has a relatively short shelf life when exposed to the environment. Thus, there hypochlorous acid has not been widely used in other environments such as in a small business or in a household to disinfect a person's skin, or to disinfect any other item in these environments such as kitchen countertops, utensils, skin contact tools such as barber razors and tattoo equipment, etc.

Thus, there is a need for a dispensing system for dispensing pure hypochlorous acid along with another substance that compliments the hypochlorous acid to disinfect and treat items in a variety of environments.

### SUMMARY

An objective of the embodiment is to fulfill the need referred to above. In accordance with an embodiment, this objective is obtained by a hypochlorous acid dispensing system having a first cartridge containing pure hypochlorous acid. A second cartridge, separate from the first cartridge, contains a substance that can be mixed with or used with the pure hypochlorous acid. A dispensing structure is associated with each of the first and second cartridges and is constructed and arranged to cause dispensing of the pure hypochlorous acid and the substance separately or simultaneously. A kit including at least one of the first cartridges and at last one of the second cartridges is also provided.

Other objectives, features and characteristics of the present embodiment, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The invention will be better understood from the following detailed description of the preferred embodiments thereof, taken in conjunction with the accompanying drawing, wherein like reference numerals refer to like parts, in which:

FIG. 1 shows a HOCl acid dispensing system in accordance with an embodiment.

FIG. 2 shows a HOCl acid dispensing system in accordance with a second embodiment.

### DETAILED DESCRIPTION OF EXAMPLE EMBODIMENTS

Hypochlorous acid (HOCl) is a typically used in industry to disinfect surfaces in restrooms and hospitals. However, if used as a disinfectant on human skin, pure HOCl acid in such a concentration could irritate the skin and would have an unsatisfactory high chlorine odor.

Applicant found that the use of pure or 100% HOCl with a chlorine concentration in the range of 5 ppm to 50 ppm with a PH preferably in a range between 4 and 7 for skin disinfecting use. For use in disinfecting on non-human objects such as surfaces, the chlorine concentration is in the range of 50-100 ppm. The HOCl can be provided in liquid or gel form. Advantageously since the pure HOCL is organic, it remains inert, stable and non-toxic.

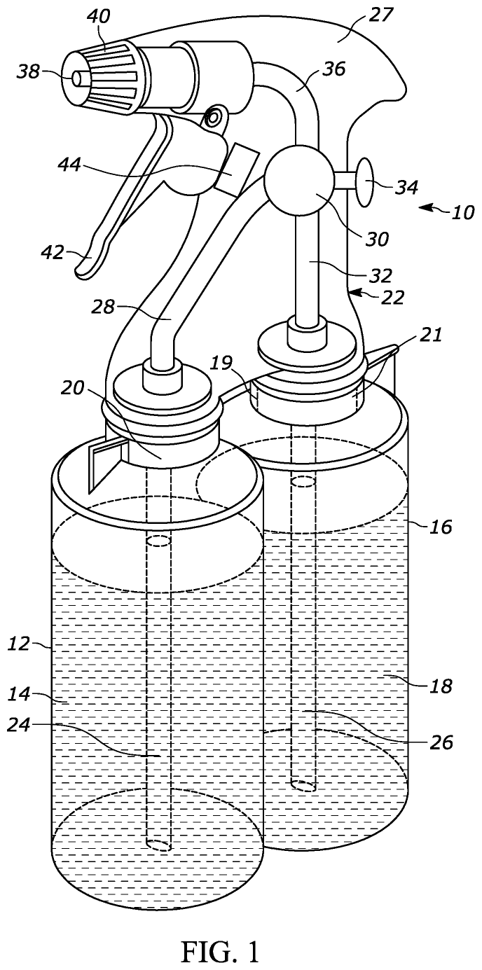

To enable such HOCl acid to be used by a typical consumer, with reference to FIG. 1, a HOCl acid dispensing system is shown, generally indicated at 10, in accordance with an embodiment of the invention. The system 10 comprises a first container or cartridge 12 containing HOCl acid 14 and a second container or cartridge 16 containing any substance 18 that can be mixed with or used with the HOCl acid 14. The terms container and cartridge can be used interchangeably and in the broadest aspect of the invention can be any reservoir that can hold a liquid, gel or a solid substance. In the embodiment, each cartridge preferably has a sealed top and has a cover (not shown) over the seal. The cover is preferably threaded onto male threads 19 on end of the cartridge. Prior to use, the seal and cover is removed and the cartridge 12, 16 is threaded into female threads in a respective container receiver 20, 21, each of which is disposed at end of a dispensing structure, generally indicated at 22.

A first suction tube 24 extends from container receiver 20 into the interior of the cartridge 12 and a second suction tube 26 extends from container receiver 21 into the interior of the cartridge 16. Within housing 27, a first intermediate tube 28, in communication with tube 24, is connected between the container receiver 20 and a valve 30 and a second intermediate tube 32, in communication with tube 26, is connected between the container receiver 21 and the valve 30. The first suction tube 24 and the first intermediate tube 28 can be considered to be a first tube structure of the system 10 and the second suction tube 26 and the second intermediate tube 32 can be considered to be a second tube structure of the system 10.

The valve 30 is constructed and arranged to restrict the flow for each intermediate tubes 28 and 32 between 0% (closed) and 100% (fully open) flow. A manually actuated control knob 34 is associated with the valve to control regulate the restriction of flow from the intermediate tubes 28 and 32. A delivery tube 36 is connected between the output of the valve 30 and an outlet or nozzle 38 of the dispensing structure 22. The nozzle 38 is housed in an adjustable nozzle head 40 which may be rotated to adjust the liquid output between a jet and a spray of liquid or gel droplets.

In the embodiment, if the valve 30 is at least partially open to each intermediate tube 28, 32, operation of a single actuator or trigger 42 causes a volume of liquid or gel to be pumped via pumping structure 44 from each cartridge 12 and 16 simultaneously via the dip tubes 24 and 26 and nozzle 38. The pumping structure 44 is shown schematically in FIG. 1 connected with the trigger 42 and may be a conventional piston/cylinder pumping mechanism of the type described in detail in U.S. Pat. No. 5,332,157, the content of which is hereby incorporated herein by reference in its entirety.

Since the adjustable valve 30 is provided, the HOCl acid 14 in cartridge 12 can be dispensed alone by closing off dispensing of the substance 18 in the second cartridge 16. In a similar manner, substance 18 in the second cartridge 16 can be dispensed alone by closing off dispensing of the HOCl acid 14 in cartridge 12. As noted above, by adjusting the valve, the HOCl acid 14 in cartridge 12 and the substance 18 in cartridge 16 can be controllably mixed and then dispensed through the nozzle 38 via the delivery tube 36.

In the system 10, one of the cartridges (in the embodiment cartridge 12) must contain HOCl acid 14. As noted above, the other cartridge 16 can contain any other substance 18 that can be mixed or used with the HOCl acid 14. For example, if the system 10 is to be used as a bodily disinfectant, the HOCl acid 14 can be used to disinfect the skin, killing bacteria. The organic HOCl acid disinfectant can be applied to bodily tissue such as underarm skin, vaginal tissue, skin folds or cavities of an obese person and acne areas on the skin. The HOCl acid disinfectant is not suitable for use in the mouth. With the above chlorine concentration, no bleaching of clothes will occur.

For deodorant use, the second cartridge 16 can contain the substance 18 in the form of a conventional antiperspirant containing, for example, aluminium chlorohydrate or aluminium-zirconium tetrachlorohydrate gly. If antiperspirant is not desired, the second cartridge 14 can contain a perfume or fragrance, a cream or liquid moisturizer, a liquid soap or other desired substance.

The second cartridge 14 can contain a medication. For example, for use in treating acne, the second cartridge 16 can contain an acne treatment substance such as retinoids, salicylic acid or glycolic acid. For treating diaper rash, the medication can be an ointment such as zinc oxide. The medication can be a steroid, or an antibiotic or any other medication that can be used externally on the human body.

The system 10 can be used in disinfecting tools/devices that contact the skin, such as razors used in barber shops, tattoo and/or piercing equipment. For example, for razor applications, the HOCl acid 14 in cartridge 12 can be applied simultaneously with or separate from alcohol or a lubricant in cartridge 16, and for tattoo and/or piercing equipment, the HOCl acid 14 in cartridge 12 can be applied simultaneously with or separate from alcohol in cartridge 16 for quick drying. Regarding tattooing or piercing, after such a procedure, the system 10 can be used by applying the HOCl acid 14 in cartridge 12 to the skin to maintain a clean and disinfected skin area. The substance 18 in the second cartridge 16 can be a skin treatment substance such as a moisturizer, petroleum jelly, an antibiotic, a liquid bandage, an adhesive to aid in a bandage adhering to skin, or other substance that promotes skin healing such as at least one of a skin cell growth substance, a scar preventing substance, aloe, zinc, vitamins A, B, D, and E or a combination thereof.

The system 10 can be used to disinfect vaping devices, hookah equipment, beverage dispensing (e.g., beer tap) equipment, as well as medical devices such as CPAP machines. For example, HOCl acid 14 in cartridge 12 can be applied to such equipment/devices as a disinfectant simultaneously with or separate from applying alcohol or other drying agent as the substance 18 dispensed from cartridge 16.

The system 10 has use in disinfecting and lubricating sex toys. For example, HOCl acid 14 in cartridge 12 can be applied to a sex toy to disinfect the toy simultaneously with or separate from applying a lubricant or alcohol to the toy as the substance dispensed from cartridge 16.

The system 10 can be used in disinfecting household areas such as the kitchen and the bathroom by applying the HOCl acid 14 in cartridge 12 to disinfect surfaces. The substance 18 in the second cartridge 16 can be a fragrance such an orange or lemon fragrance to provide a “clean” odor. Instead of using dispenser structure 22 of FIG. 1, the two cartridges 14, 16 can be employed in a plug-in type dispenser structure that plugs into an electrical socket, such as disclosed in U.S. Pat. No. 6,5770,72, the content of which is hereby incorporated by reference into this specification. This type of dispenser structure would need to be modified to accept and dispense two cartridges simultaneously, namely a first cartridge containing the HOCl acid 14 and a second cartridge containing a fragrance.

For best shelf life of the pure HOCl acid 14, the cartridge 12 can be of opaque PET material to prevent light penetration. It is noted that FIG. 1 shows transparent cartridges 12, 16 for illustration clarity. If the cartridge 12 remains unopened, the shelf life can be up to one year. A kit of preferably 3, 6, or 12 cartridges 12 of the HOCl acid 14 can be provided to the consumer. Also, a kit of various cartridges 12 and 16 can be provided. For example, the kit can contain one or more HOCl acid cartridges 12, and a variety of cartridges 16, each containing a different substance. For example, an antiperspirant, a moisturizer, petroleum jelly, a soap, a perfume or fragrance, or a skin healing or acne treatment substance can be provided in cartridge 16.

Although an example of a dispenser structure 22 is disclosed for non-pressurized cartridges 12 and 16, it can be appreciated that the capsules can be pressurized and be dispensed by appropriate dispensing structure such as those disclosed in U.S. Pat. No. 10,351,334, the content of which is hereby incorporated by reference into this specification. Also, the dispenser structure 22 can be an electrostatic-type sprayer.

Another embodiment can provide the cartridge or container 12 containing liquid or gel HOCl acid 14 together with the second cartridge or container 16 containing a solid, such as a deodorant or antiperspirant stick, as the substance 18. The HOCl acid 14 can be dispensed on the skin simultaneously with or separate from the solid of the stick. An example of dispensing structure for such containers 12, 16 can be of the type disclosed in U.S. Pat. No. 8,240,930, the content of which is hereby incorporated by reference into this specification. Alternatively, to apply the HOCl acid 14 and solid substance 18 simultaneously, the dispenser structure can be configured to cause a portion of the solid in cartridge 16 to pass through or be soaked with the HOCl acid disposed in a separate reservoir.

Although the dispensing structure 22 is disclosed as spraying the contends from cartridges 12, 16, a dispensing structure can be configured to cause the content of the cartridges 12, 16 to be rolled-on, bushed on dripped on or otherwise dispensed.

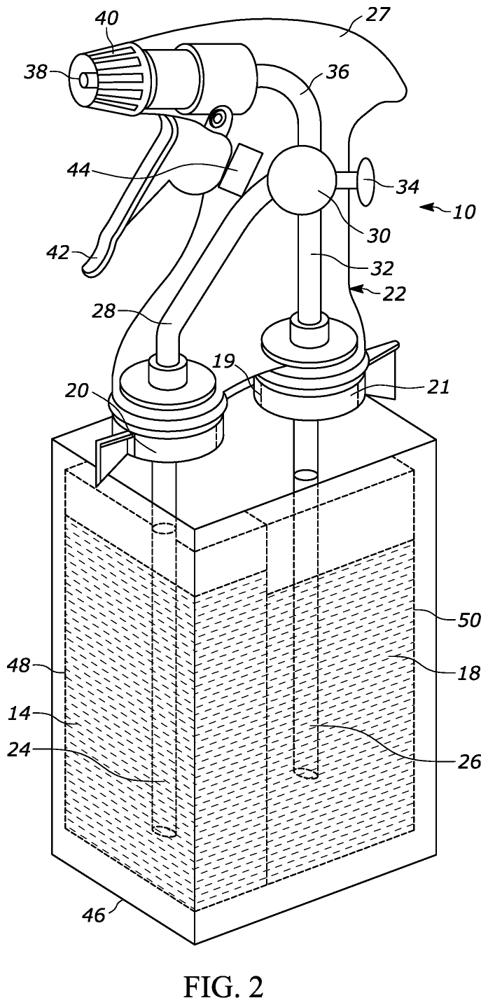

With reference to FIG. 2, instead of employing two separate cartridges 12, 16, a single container 46 can be provided having two separate, internal compartments. The first compartment 48 can contain the HOCl acid 14 and the second compartment 50 can contain any substance 18 that can be mixed with or used with the HOCl acid 14. The container 46 can be configured to accommodate the dispenser structure 22 for dispensing the HOCl acid 14 alone or simultaneously with substance 18.

In yet another application, the HOCl acid 14 in cartridge 12 can be sprayed or applied onto a growing consumable plant such as a marijuana plant, tobacco or on other plants that are not consumable but that produce a consumable fruit or vegetable. Spraying can include delivering a fog or a mist. The substance 18 in cartridge 16 can be a plant fertilizer or plant growth hormones, or a in some cases, an insecticide.

Thus, the embodiment provides an effective way to disinfect skin or other surfaces by employing organic HOCl acid 14 which is 100% effective against all pathogens in combination with a wide variety of complimentary substances.

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the following claims.

What is claimed is:

1. A hypochlorous acid dispensing system comprising:
a first cartridge containing pure hypochlorous acid;
a second cartridge, separate from the first cartridge, containing a substance that can be mixed with or used with the pure hypochlorous acid; and
dispensing structure associated with each of the first and second cartridges and constructed and arranged to cause dispensing of the pure hypochlorous acid and the substance separately or simultaneously.

2. The system of claim 1, wherein the pure hypochlorous acid has a chlorine concentration in a range of 5 ppm to 50 ppm and a pH in a range of 4 to 7.

3. The system of claim 1, wherein the pure hypochlorous acid has a chlorine concentration in a range of 50 ppm to 100 ppm and a pH in a range of 4 to 7.

4. The system of claim 1, wherein the dispensing structure is constructed and arranged to cause mixing of the pure hypochlorous acid with the substance.

5. The system of claim 1, wherein the dispensing structure comprises:
first tube structure having one end disposed in the first cartridge and another end connected with a valve,
second tube structure having one end disposed in the second cartridge and another end connected with the valve,
a delivery tube connected to an outlet of the valve,
a nozzle defining an outlet, the nozzle being connected to the delivery tube, and
an actuator and pumping structure constructed and arranged to cause the pure hypochlorous acid and/or the substance be drawn through the valve and the delivery tube to be dispensed through the nozzle.

6. The system of claim 1, wherein the substance in the second cartridge is an antiperspirant, a moisturizer, a soap, petroleum jelly, zinc oxide, alcohol, a lubricant, a fragrance, a skin treatment substance, a fertilizer, a growth hormone, an insecticide, or a medication.

7. The system of claim 6, wherein the skin treatment substance includes at least one of aloe, zinc, vitamins, a skin cell growth substance, and a scar preventing substance, a liquid bandage and an adhesive.

8. The system of claim 6, wherein the medication includes at least one of an antibiotic, a steroid, a retinoid, a salicylic acid, a glycolic acid,

[symbol]

9. A kit comprising:
at least one first cartridge containing pure hypochlorous acid; and
at least one a second cartridge, separate from the first cartridge, containing a substance that can be mixed with or used with the pure hypochlorous acid.

10. The kit of claim 9, wherein the pure hypochlorous acid has a chlorine concentration in a range of 5 ppm to 50 ppm and a pH in a range of 4 to 7.

11. The kit of claim 9, wherein the pure hypochlorous acid has a chlorine concentration in a range of 50 ppm to 100 ppm and a pH in a range of 4 to 7.

12. The kit of claim 9, wherein the substance in the second cartridge is an antiperspirant, a moisturizer, a soap, petroleum jelly, zinc oxide, alcohol, a lubricant, a fragrance, a skin treatment substance, a fertilizer, a growth hormone, an insecticide, or a medication.

13. The kit of claim 12, wherein the skin treatment substance includes at least one of aloe, zinc, vitamins, a skin cell growth substance, and a scar preventing substance, a liquid bandage and an adhesive.

14. The kit of claim 12, wherein the medication includes at least one of an antibiotic, a steroid, a retinoid, a salicylic acid, a glycolic acid,

15. The kit of claim 9, wherein each cartridge is non-pressurized.

16. A hypochlorous acid dispensing system comprising:
a container having a first compartment containing pure hypochlorous acid and a second container, separate from the first container, containing a substance that can be mixed with or used with the pure hypochlorous acid; and
dispensing structure associated with the container and thus with each of the first and second compartments and constructed and arranged to cause dispensing of the pure hypochlorous acid and the substance separately or simultaneously.

17. The system of claim 16, wherein the pure hypochlorous acid has a chlorine concentration in a range of 5 ppm to 50 ppm and a pH in a range of 4 to 7.

18. The system of claim 16, wherein the pure hypochlorous acid has a chlorine concentration in a range of 50 ppm to 100 ppm and a pH in a range of 4 to 7.

19. The system of claim 16, wherein the dispensing structure is constructed and arranged to cause mixing of the pure hypochlorous acid with the substance.

20. The system of claim 16, wherein the substance in the second cartridge is an antiperspirant, a moisturizer, a soap, petroleum jelly, zinc oxide, alcohol, a lubricant, a fragrance, a skin treatment substance, a fertilizer, a growth hormone, an insecticide, or a medication.
